(19) United States
(12) Patent Application Publication
(10) Pub. No.: US 2008/0063640 A1
(43) Pub. Date: Mar. 13, 2008

(54) Pin-Prc Transition Genes

(75) Inventors: Yusuke Nakamura, Kanagawa (JP); Hidewaki Nakagawa, Tokyo (JP); Shuichi Nakatsuru, Saitama (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Oncotherapy Science, Inc., Kawasaki-shi (JP)

(21) Appl. No.: 10/598,296

(22) PCT Filed: Feb. 4, 2005
(86) PCT No.: PCT/JP05/02090

§ 371 Date: Jun. 11, 2007

Related U.S. Application Data
(60) Provisional application No. 60/548,335, filed on Feb. 27, 2004.

(51) Int. Cl. A61K 31/70; A61K 39/00; A61K 39/395; A61P 13/08; C12Q 1/68; C40B 30/04; C40B 30/06; C40B 40/08

(52) U.S. Cl. 424/138.1; 424/277.1; 435/6; 506/10; 506/17; 506/9; 514/44

(57) ABSTRACT

Objective methods for diagnosing a predisposition to developing prostate cancer (PRC) are described herein. In one embodiment, the diagnostic method involves the determining a expression level of PRC-associated gene that discriminate between PRC and PIN. The present invention further provides methods of screening for therapeutic agents useful in the treatment of PRC, methods of treating PRC.

This application claims the benefit of U.S. Provisional Application Ser. No. 60/548,335 filed Feb. 27, 2004, the contents of which are hereby incorporated by reference in its entirety.

### TECHNICAL FIELD

The present invention relates to methods of detecting and diagnosing a predisposition to developing prostate cancer (PRC) as well as methods of treating and preventing prostate cancer.

### BACKGROUND ART

Prostate cancer (PRC) is one of the most common malignancy in males and the second-leading cause of cancer-related deaths in the United States and Europe (Gronberg et al., 2003). The testing for prostate specific antigen (PSA) in serum can detect early stage of PRC and it is now a gold standard to screen PRC in the high-risk population.

Incidence of prostate cancer is increasing steadily in developed countries according to the prevalence of Western-style diet and increasing number of senior population. Early diagnosis through serum testing for prostate specific antigen (PSA) provides an opportunity for curative surgery and has significantly improved the prognosis of prostate cancer, but up to 30% of patients treated with radical prostatectomy relapse their cancer (Han et al., 2001). Most relapsed or advanced cancers respond to androgen ablation therapy because prostate cancer growth is initially androgen-dependent. However, they eventually progress to androgen-independent disease, at which point they are no longer responsive to androgen ablation therapy. The most serious clinical problem of prostate cancer is that androgen-independent prostate cancer is unresponsive to any other therapies (Gronberg, 2003), and establishing new therapies other than androgen ablation therapy against prostate cancer are a urgent issue for management of prostate cancer.

High-grade prostatic intraepithelial neoplasia (PIN) is widely accepted as the main premalignant lesion without invasion of the basal membrane of the acini, which has the potential to progress to invasive PRC (McNeal and Bostwick et al. 1986, DeMarzo et al. 2003, Abate-Shen et al. 2000, Montironi et al. 2002,). PIN does not significantly elevate serum PSA concentration and cannot be detected by ultrasound.

High-grade PIN has a high predictive value as a marker for PRC, and its identification warrants repeat biopsy for concurrent or subsequent invasive PRC. Only prostate needle biopsy can recognize this minimal lesions and its identification warrants repeat biopsy for concurrent or subsequent invasive PRC (Bostwick 2000). Performing saturation prostate biopsies to rule out any coexistent prostate cancer followed by every 3-6 month serial repeated prostate biopsies is currently the only way in which to manage patients found to have high-grade PIN. But the reliability of this diagnosis is highly dependent on the technique of prostate needle biopsy, histological processing, and experience of reviewing pathologists (van der Kwast et al. 2003). They cannot perfectly discriminate PRC lesions from PRC nor identify the patients with invasive PRC among the high-risk people with PINs.

Hence accurate identification of PINs and PRC and understanding the prostatic carcinogenesis through PINs are important to avoid error in the diagnosis of invasive PRC and in patient management (Steiner 2001). However, the natural history of PINs and molecular mechanism of the putative transition form PINs to PRC reminds unclear and it is still controversial whether these PIN lesions without PRC should be treated or not.

cDNA microarray technologies have enabled to obtain comprehensive profiles of gene expression in normal and malignant cells, and compare the gene expression in malignant and corresponding normal cells (Okabe et al., Cancer Res 61:2129-37 (2001);. Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnesyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al., Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically over-expressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in ⁵¹Cr-release assays (Kawano et al., Cance Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Histocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

### SUMMARY OF THE INVENTION

The gene-expression profiles of cancer cells from 20 PRCs and 10 high-grade PINs were analyzed using cDNA microarray representing 23,040 genes coupled with laser microbeam microdissection (LMM) to characterize the molecular mechanisms involved in the putative transition from PINs to invasive PRC. By comparing expression patterns between cancer cells from diagnostic PRC patients and PIN cells purely selected with Laser Microdisection, 40 genes were identified as being up-regulated in PRC cells compared to in PIN cells, and 98 genes were identified as being down-regulated in PRC cells compared to in PIN cells. In addition, selection was made of candidate molecular markers with the potential of detecting cancer-related proteins in serum or sputum of patients, and discovered some potential targets for development of signal-suppressing strategies in human PRC.

Laser microdissection allows us to isolate pure cell populations and enables the precise evaluation (Emmert-Buck et al., 1996). Once isolated high-grade PINs without PRC are identified, treatment of high-grade PINs would appear to be of clinical benefit, and preventing from PINs to invasive PRC would reduce morbidity, enhance the quality of life, delay surgery or radiation, and increase the interval for surveillance requiring invasive procedures (Steiner et al. 2001, Nelson et al. 2001, Montironi et al. 2002). These data would provide important information on prostatic carcinogenesis and would be greatly useful to identify candidate genes whose products can be targeted for drug design for treatment and prevention of PRC.

The present invention is based on the discovery of a pattern of gene expression correlated with PRC and PIN. The genes that are differentially expressed in PRC compared to PIN are collectively referred to herein as “PRC nucleic acids” or “PRC polynucleotides” and the corresponding encoded polypeptides are referred to as “PRC polypeptides” or “PRC proteins.”

Accordingly, the present invention features a method of diagnosing or determining a predisposition to developing PRC in a subject by determining an expression level of a PRC-associated gene in a patient derived biological sample, such as tissue sample. By PRC associated gene is meant a gene that is characterized by an expression level which differs in a PRC cell compared to PIN cell. A PRC-associated gene includes for example PRC 1-138. An alteration, e.g., increase or decrease of the level of expression of the gene compared to expression level of the gene in PIN indicates that the subject is at risk of developing PRC.

In the context of the present invention, the phrase “control level” refers to a protein expression level detected in a control sample and includes both a normal control level and an prostate cancer control level. A control level can be a single expression pattern derived from a single reference population or from a plurality of expression patterns. For example, the control level can be a database of expression patterns from previously tested cells. A “normal control level” refers to a level of gene expression detected in a normal, healthy individual or in a population of individuals known not to be suffering from prostate cancer. A normal individual is one with no clinical symptoms of PRC and PIN. On the other hand, a “PRC control level” refers to an expression profile of PRC-associated genes found in a population suffering from PRC.

An increase in the expression level of one or more PRC 1-40 detected in a test sample as compared to a level in PIN indicates that the subject (from which the sample was obtained) suffers from or is at risk of developing PRC. In contrast, a decrease in the expression level of one or more PRC 41-138 detected in a test sample as compared to a level in PIN indicates that said subject suffers from or is at risk of developing PRC.

Alternatively, expression of a panel of PRC-associated genes in a sample can be compared to a PRC level of the same panel of genes. A similarity between a sample expression and PRC control expression indicates that the subject (from which the sample was obtained) suffers from or is at risk of developing PRC. By PRC level is meant the expression profile of the PRC-associated genes found in a population suffering from PRC.

According to the present invention, gene expression level is deemed “altered” when gene expression is increased or decreased 10%, 25%, 50% as compared to the level in PIN. Alternately, the gene expression may be also be deemed to be altered if gene expression is increased or decreased 1, 2, 5 or more fold as compared to the level in PIN. Expression is determined by detecting hybridization, e.g., on an array, of a PRC-associated gene probe to a gene transcript of the patient-derived tissue sample.

In the context of the present invention, the patient derived tissue sample is any tissue obtained from a test subject, e.g., a patient known to or suspected of having PRC. For example, the tissue may contains an epithelial cell. More particularly, the tissue may be an epithelial cell from prostate tissue.

The present invention provides method for discriminating PRC form PINs and detect malignant PRC cells with high sensitivity using PRC 1-138. Especially, APOD is useful as specific markers for discriminating PRC from high-grade PINs.

The present invention also provides a PRC reference expression profile, comprising a gene expression level of two or more of PRC 1-138. Alternatively, the present invention provides a PRC reference expression profile may comprise the levels of expression of two or more up PRC 1-40 or PRC 41-138.

The present invention further provides methods of identifying an agent that inhibits or enhances the expression or activity of a PRC-associated gene, e.g. PRC 1-138 by contacting a test cell expressing a PRC-associated gene with a test agent and determining the expression level or activity of the PRC associated gene or the biological activity of its gene product. The test cell may be an epithelial cell, such as an epithelial cell obtained from prostate tissue. A decrease in the expression level of PRC-associated gene or biological activity its gene product as compared to that of the up-regulated marker gene in PRC or gene product indicates that the test agent is an inhibitor of expression or function of the PRC-associated gene and may be used to reduces a symptom of PRC, e.g., the expression of one or more PRC 1-40. Alternatively, an increase in the expression level of PRC-associated gene or biological activity its gene product as compared to that of the down-regulated marker gene in PRC or gene product indicates that said test agent is an enhancer of expression or function of the PRC associated gene and may be used to reduces a symptom of PRC, e.g., the expression of one or more PRC 41-138. Moreover, a decrease of the expression level or biological activity in the presence of the agent compared to that in the absence of the test agent indicates the agent is an inhibitor of an PRC associated up-regulated gene and useful to inhibit PRC. Alternatively, an increase of the expression level or biological activity of the PRC-associated gene compared to that in the absence of the test agent indicates that the test agent augments expression or activity of the down-regulated PRC associated gene.

The present invention also provides a kit comprising a detection reagent which binds to two or more PRC polynucleotides or PRC polypeptides. Also provided is an array of nucleic acids that binds to two or more PRC nucleic acids.

The lists of the genes associated with malignant transformation also could provide with a number of information which is essential to establish novel chemo-preventive drugs for PRC transformation, and these chemo-preventive drugs could be treated effectively to the selected high-risk population of PRC, that is, those with high-grade PINs for the purpose of treating or preventing PRC.

Therapeutic methods of the present invention include a method of treating or preventing PRC in a subject including the step of by administering to the subject an antisense composition. In the context of the present invention, the antisense composition reduces the expression of the specific target gene. For example, the antisense composition may contain a nucleotide, which is complementary to PRC-associated gene sequence selected from the group consisting of PRC 1-40. Alternatively, the present method may include the steps of administering to a subject a small interfering RNA (siRNA) composition. In the context of the present invention, the siRNA composition reduces the expression of a PRC nucleic acid selected from the group consisting of the PRC 1-40. In yet another method, the treatment or prevention of PRC in a subject may be carried out by administering to a subject a ribozyme composition. In the context of the present invention, the nucleic acid-specific ribozyme composition reduces the expression of a PRC nucleic acid selected from the group consisting of the PRC 1-40. Other therapeutic methods include those in which a subject is administered a compound that increases the expression of one or more of the PRC 41-138 or the activity of a polypeptide encoded by one or more of the PRC 41-138. Furthermore, PRC can be treated by administering a protein encoded by PRC 41-138. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art.

The present invention also includes vaccines and vaccination methods. For example, a method of treating or preventing PRC in a subject may involve administering to the subject a vaccine containing a polypeptide encoded by a nucleic acid selected from the group consisting of PRC 1-40 or an immunologically active fragment such a polypeptide. In the context of the present invention, an immunologically active fragment is a polypeptide that is shorter in length than the full-length naturally-occurring protein yet and which induces an immune response analogous to that induced by the full-length protein. For example, an immunologically active fragment should be at least 8 residues in length and capable of stimulating an immune cell such as a T cell or a B cell. Immune cell stimulation can be measured by detecting cell proliferation, elaboration of cytokines (e.g., IL-2), or production of an antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference herein in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms. Other features and advantages of the invention will be apparent from the following detailed description, and from the claims.

### BRIEF DESCRIPTION OF THE FIGURES

FIG. 1 is an illustration depicting the pathway for human prostate cancer progression. High-grade prostatic intraepithelial neoplasia (PIN) is widely accepted as the main premalignant lesion, which has the potential to progress to invasive PRC.

FIG. 2 are photographs showing the results of immunohistochemical analysis of genes that were identified to be differentially expressed in the transition from PIN to PRC. Apolipoprotein D (APOD) was abundantly expressed in PRC cells, while PINs and normal prostatic epithelium (N) from the same patient showed no expression of APOD protein. The PRC, PIN and normal prostate epithelium were included on one prostate cancer tissue. Magnification, ×200.

### DISCLOSURE OF THE INVENTION

The words “a”, “an” and “the” as used herein mean “at least one” unless otherwise specifically indicated.

The present invention is based, in part, on the discovery of changes in expression patterns of multiple nucleic acids between normal epithelial cells and PIN cells of patients known to or suspected of having PRC. Furthermore, the present invention is based, in part, on the discovery of changes in expression patterns of multiple nucleic acids between normal epithelial cells and carcinomas of patients with PRC. These expression patterns are compared and differently expressed genes were identified using a comprehensive cDNA microarray system.

The differentially expressed genes identified herein are used for diagnostic purposes as markers of predisposition to developing PRC and as gene targets, the expression of which is altered to treat or alleviate a symptom of PRC. The term “predisposition” as used herein indicates a potential to develop PRC from PIN. Predisposition can be diagnosed by measuring the expression levels of Prc-associated genes which expression level are altered in the transition from PIN to PRC.

Alternatively, the differentially expressed between PIN and PRC identified herein find diagnostic utility as markers for distinguishing PRC from PIN and as PRC gene targets, the expression of which may be altered to treat or alleviate a symptom of PRC.

The genes whose expression level is modulated (i.e., increased or decreased) in PRC patients are summarized in Tables 1 and 2, and are collectively referred to herein as “PRC-associated genes ”, “PRC nucleic acids” or “PRC polynucleotides” and the corresponding encoded polypeptides are referred to as “PRC polypeptides” or “PRC proteins.” Unless indicated otherwise, “PRC” refers to any of the sequences disclosed herein. (e.g., PRC 1-138). The genes that have been previously described are presented along with a database accession number.

By measuring expression of the various genes in a sample of cells, PRC can be diagnosed. Similarly, measuring the expression of these genes in response to various agents can identify agents for treating PRC.

The present invention involves determining (e.g., measuring) the expression of at least one, and up to all the PRC-associated genes listed in Tables 1 and 2. Using sequence information provided by the GeneBank™ database entries for known sequences, the PRC associated genes can be detected and measured using techniques well known to one of ordinary skill in the art. For example, sequences within the sequence database entries corresponding to PRC-associated genes, can be used to construct probes for detecting RNA sequences corresponding to PRC-associated genes in, e.g., Northern blot hybridization analyses. Probes typically include at least 10, at least 20, at least 50, at least 100, at least 200 nucleotides of a reference sequence. As another example, the sequences can be used to construct primers for specifically amplifying the PRC nucleic acid in, e.g., amplification-based detection methods such as reverse-transcription based polymerase chain reaction.

Expression level of one or more of PRC-associated genes in a test cell population, e.g., a patient-derived tissues sample, is then compared to the expression level(s) of the same gene(s) in a reference population. The reference cell population includes one or more cells for which the compared parameter is known, i.e., PIN cells. The expression level of PRC 1-138 in the specimens from the test cell population and reference cell population may be determined at the same time. Alternatively, expression levels of the PRC 1-138 in reference cell population can be determined by a statistical method based on the results obtained by analyzing the expression level of the gene in specimens previously collected prostate ductal carcinoma cells (e.g., PRC cells) or normal prostate ductal epithelial cells (e.g., non-PRC cells).

Whether or not a pattern of gene expression in a test cell population as compared to a reference cell population indicates a predisposition to developing PRC. For example, non-PRC cells can be used as the reference cell population. When the expression level of the gene in a test cell population does not fall within the range of reference cell population, the subject is judged to have high risk to develop PRC.

Moreover, if the reference cell population is made up of PRC cells, a similarity in gene expression profile between the test cell population and the reference cell population indicates that the test cell population includes PRC cells.

A level of expression of a PRC marker gene in a test cell population is considered “altered” if it varies from the expression level of the corresponding PRC marker gene in a reference cell population by more than 1.1, more than 1.5, more than 2.0, more than 5.0, more than 10.0 or more fold.

Differential gene expression between a test cell population and a reference cell population can be normalized to a control nucleic acid, e.g. a housekeeping gene. For example, a control nucleic acid is one which is known not to differ depending on the cancerous or non-cancerous state of the cell. The expression level of a control nucleic acid in the test and reference population can be used to normalize signal levels in the test and reference populations. Exemplary control genes include, but are not limited to, e.g., β-actin, glyceraldehyde 3-phosphate dehydrogenase and ribosomal protein P1.

The test cell population can be compared to multiple reference cell populations. Each of the multiple reference populations may differ in the known parameter. Thus, a test cell population may be compared to a first reference cell population known to contain, e.g., PRC cells, as well as a second reference population known to contain, e.g., PIN cells. The test cell may be included in a tissue type or cell sample from a subject known to contain, or suspected of containing, PRC cells.

The test cell is obtained from a bodily tissue or a bodily fluid, e.g., biological fluid (such as blood or sputum, for example). For example, the test cell may be purified from prostate tissue. Preferably, the test cell population comprises an epithelial cell. The epithelial cell is preferably from a tissue known to be or suspected to be cancerous. Cells in the reference cell population should be derived from a tissue type similar that of the to test cell. Optionally, the reference cell population is a cell line, e.g. a PRC cell line (i.e., a positive control) or a normal PIN cell line (i.e., a negative control). Alternatively, the control cell population may be derived from a database of molecular information derived from cells for which the assayed parameter or condition is known.

The subject is preferably a mammal. Exemplary mammals include, but are not limited to, e.g., a human, non-human primate, mouse, rat, dog, cat, horse, or cow.

Expression of the genes disclosed herein can be determined at the protein or nucleic acid level using methods known in the art. For example, Northern hybridization analysis using probes which specifically recognize one or more of these nucleic acid sequences can be used to determine gene expression. Alternatively, gene expression may be measured using reverse-transcription-based PCR assays, e.g., using primers specific for the differentially expressed gene sequences. Expression may also be determined at the protein level, i.e., by measuring the level of a polypeptides encoded by a gene described herein, or biological activity thereof. Such methods are well known in the art and include, but are not limited to, e.g., immunoassays that utilize antibodies to proteins encoded by the genes. The biological activities of the proteins encoded by the genes are generally well known.

### Diagnosing PRC

In the context of the present invention, PRC is diagnosed by measuring the expression level of one or more PRC polynucleotides from a test population of cells, (i.e., a patient-derived biological sample). Preferably, the test cell population contains an epithelial cell, e.g., a cell obtained from prostate tissue. Gene expression can also be measured from blood or other bodily fluids such as urine. Other biological samples can be used for measuring protein levels. For example, the protein level in blood or serum derived from a subject to be diagnosed can be measured by immunoassay or other conventional biological assay.

Expression of one or more of an PRC-associated genes, e.g., PRC 1-138 is determined in the test cell or biological sample and compared to the normal control expression level associated with the one or more PRC-associated gene(s) assayed. A normal control level is an expression profile of a PRC-associated gene typically found in a population known not to be suffering from PRC. An alteration (e.g., an increase or a decrease) in the level of expression in the patient-derived tissue sample of one or more PRC associated gene indicates that the subject is suffering from or is at risk of developing PRC. For example, an increase in the expression of one or more up-regulated PRC-associated genes, PRC 1-40, in the test population as compared to the expression level in PIN indicates that the subject is suffering from or is at risk of developing PRC. Conversely, a decrease in expression of one or more down-regulated PRC-associated genes, PRC 41-138, in the test population compared to the expression level in PIN indicates that the subject is suffering from or is at risk of developing PRC.

Alteration of one or more of the PRC-associated genes in the test population as compared to the expression level in PIN indicates that the subject suffers from or is at risk of developing PRC. For example, alteration of at least 1%, at least 5%, at least 25%, at least 50%, at least 60%, at least 80%, at least 90% or more of the panel of PRC-associated genes (PRC 1-40 or PRC 41-138) indicates that the subject suffers from or is at risk of developing PRC.

The expression levels of the PRC 1-138 in a particular specimen can be estimated by quantifying mRNA corresponding to or protein encoded by PRC 1-138. Quantification methods for MRNA are known to those skilled in the art. For example, the levels of mRNAs corresponding to the PRC 1-138 can be estimated by Northern blotting or RT-PCR. Since the nucleotide sequence of the PRC 1-138 have already been reported. Anyone skilled in the art can design the nucleotide sequences for probes or primers to quantify the PRC 1-138.

Also the expression level of the PRC 1-138 can be analyzed based on the activity or quantity of protein encoded by the gene. A method for determining the quantity of the PRC 1-138 protein is shown in bellow. For example, immunoassay method is useful for the determination of the proteins in biological materials. Any biological materials can be used for the determination of the protein or it's activity. For example, blood sample is analyzed for estimation of the protein encoded by a serum marker. On the other hand, a suitable method can be selected for the determination of the activity of a protein encoded by the PRC 1-138 according to the activity of each protein to be analyzed.

In the present invention, a diagnostic agent for diagnosing predisposition to developing PRC, is also provided. The diagnostic agent of the present invention comprises a compound that binds to a polynucleotide or a polypeptide of the present invention. Preferably, an oligonucleotide that hybridizes to the polynucleotide of the PRC 1-40, or an antibody that binds to the polypeptide of the PRC 1-40 may be used as such a compound.

### Identifying Agents That Inhibit or Enhance PRC-Associated Gene Expression

An agent that inhibits the expression or activity of a PRC-associated gene or the activity of its gene product can be identified by contacting a test cell population expressing an PRC-associated up-regulated gene with a test agent and then determining the expression level or activity of the PRC-associated gene. A decrease in the level of expression or activity of the PRC-associated gene or in the level of activity of its gene product in the presence of the agent as compared to the expression or activity in the absence of the test agent indicates that the agent is an inhibitor of a PRC associated up-regulated gene and useful in inhibiting PRC.

Alternatively, an agent that enhances the expression of an PRC-associated down-regulated gene or the activity of its gene product can be identified by contacting a test cell population expressing a PRC associated gene with a test agent and then determining the expression level or activity of the PRC-associated down-regulated gene. An increase in the level of expression of the PRC-associated gene or in the level of activity of its gene products as compared to the expression or activity in the absence of the test agent indicates that the test agent augments expression of PRC-associated down-regulated gene or activity of its gene product.

The test cell population may be any cell expressing the PRC-associated genes. For example, the test cell population may contain an epithelial cell, such as a cell derived from prostate tissue. Furthermore, the test cell may be an immortalized cell line derived from a PRC cell. Alternatively, the test cell may be a cell which has been transfected with a PRC-associated gene or which has been transfected with a regulatory sequence (e.g. promoter sequence) from a PRC-associated gene operably linked to a reporter gene.

### Assessing Efficacy of Treatment of PRC in a Subject

The differentially expressed Prc-associated genes identified herein also allow for the course of treatment of PRC to be monitored. In this method, a test cell population is provided from a subject undergoing treatment for PRC. If desired, test cell populations are obtained from the subject at various time points, before, during, and/or after treatment. Expression of one or more of the Prc-associated genes, in the cell population is then determined and compared to a reference cell population which includes cells whose PRC state is known. In the context of the present invention, the reference cells should have not been exposed to the treatment of interest.

If the reference cell population contains no PRC cells, a similarity in the expression of a PRC-associated gene in the test cell population and the reference cell population indicates that the treatment of interest is efficacious. However, a difference in the expression of a PRC-associated gene in the test population and PIN reference cell population indicates a less favorable clinical outcome or prognosis. Similarly, if the reference cell population contains PRC cells, a difference between the expression of a PRC-associated gene in the test cell population and the reference cell population indicates that the treatment of interest is efficacious, while a similarity in the expression of a PRC-associated gene in the test population and the reference cell population indicates a less favorable clinical outcome or prognosis.

Additionally, the expression level of one or more PRC-associated genes determined in a subject-derived biological sample obtained after treatment (i.e., post-treatment levels) can be compared to the expression level of the one or more PRC-associated genes determined in a subject-derived biological sample obtained prior to treatment onset (i.e., pre-treatment levels). If the PRC-associated gene is an up-regulated gene, a decrease in the expression level in a post-treatment sample indicates that the treatment of interest is efficacious while an increase or maintenance in the expression level in the post-treatment sample indicates a less favorable clinical outcome or prognosis. Conversely, if the PRC-associated gene is an down-regulated gene, an increase in the expression level in a post-treatment sample may indicate that the treatment of interest is efficacious while a decrease or maintenance in the expression level in the post-treatment sample indicates a less favorable clinical outcome or prognosis.

As used herein, the term “efficacious” indicates that the treatment leads to a reduction in the expression of a pathologically up-regulated gene, an increase in the expression of a pathologically down-regulated gene or a decrease in size, prevalence, or metastatic potential of PRC in a subject. When a treatment of interest is applied prophylactically, the term “efficacious” means that the treatment retards or prevents a PRC from forming or retards, prevents, or alleviates a symptom of clinical PRC. Assessment of prostate tumors can be made using standard clinical protocols.

In addition, efficaciousness can be determined in association with any known method for diagnosing or treating PRC. PRC can be diagnosed, for example, by identifying symptomatic anomalies, e.g., weight loss, abdominal pain, back pain, anorexia, nausea, vomiting and generalized malaise, weakness, and jaundice.

### Selecting a Therapeutic Agent for Treating PRC That is Appropriate for a Particular Individual

Differences in the genetic makeup of individuals can result in differences in their relative abilities to metabolize various drugs. An agent that is metabolized in a subject to act as an anti-PRC agent can manifest itself by inducing a change in a gene expression pattern in the subject's cells from that characteristic of a cancerous state to a gene expression pattern characteristic of a non-cancerous state. Accordingly, the differentially expressed PRC-associated genes disclosed herein allow for a putative therapeutic or prophylactic inhibitor of PRC to be tested in a test cell population from a selected subject in order to determine if the agent is a suitable inhibitor of PRC in the subject.

To identify an inhibitor of PRC, that is appropriate for a specific subject, a test cell population from the subject is exposed to a therapeutic agent, and the expression of one or more of PRC 1-138 genes is determined.

In the context of the method of the present invention, the test cell population contains a PRC cell expressing a PRC-associated gene. Preferably, the test cell is an epithelial cell. For example a test cell population may be incubated in the presence of a candidate agent and the pattern of gene expression of the test sample may be measured and compared to one or more reference profiles, e.g., PIN reference expression profile.

A decrease in expression of one or more of PRC 1-40 or an increase in expression of one or more of PRC 41-138 in a test cell population relative to a reference cell population containing PRC indicates that the agent has therapeutic potential.

In the context of the present invention, the test agent can be any compound or composition. Exemplary, the test agents include, but are not limited to, immunomodulatory agents.

### Screening Assays for Identifying Therapeutic Agents

The differentially expressed PRC-associated genes disclosed herein can also be used to identify candidate therapeutic agents for treating PRC. The method of the present invention involves screening a candidate therapeutic agent to determine if it can convert an expression profile of one or more PRC-associated genes, such as PRC 1-138 characteristic of a PRC state to a gene expression pattern characteristic of a PIN state.

In the present invention, PRC 1-138 are useful for screening of therapeutic agent for treating or preventing PRC.

In the instant method, a cell is exposed to a test agent or a plurality of test agents (sequentially or in combination) and the expression of one or more PRC 1-138 in the cell is measured. The expression profile of the PRC-associated gene(s) assayed in the test population is compared to expression level of the same PRC-associated gene(s) in a reference cell population that is not exposed to the test agent.

An agent capable of stimulating the expression of an under-expressed gene or suppressing the expression of an overexpressed genes has potential clinical benefit. Such agents may be further tested for the ability to prevent PRC in animals or test subjects.

In a further embodiment, the present invention provides methods for screening candidate agents which are potential targets in the treatment of PRC. As discussed in detail above, by controlling the expression levels of marker genes or the activities of their gene products, one can control the onset and progression of PRC. Thus, candidate agents, which are potential targets in the treatment of PRC, can be identified through screening methods that use such expression levels and activities of as indices of the cancerous or non-cancerous state. In the context of the present invention, such screening may comprise, for example, the following steps:
a) contacting a test compound with a polypeptide encoded by a polynucleotide selected from the group consisting of PRC 1-138,

b) detecting the binding activity between the polypeptide and the test compound; and
c) selecting the test compound that binds to the polypeptide.

Alternatively, the screening method of the present invention may comprise the following steps:
a) contacting a candidate compound with a cell expressing one or more marker genes, wherein the one or more marker genes are selected from the group consisting of PRC 1-138; and
b) selecting the candidate compound that reduces the expression level of one or more marker genes selected from the group consisting of PRC 14, or elevates the expression level of one or more marker genes selected from the group consisting of PRC 41-138.

### Cells expressing a marker gene include, for example, cell lines established from PRC; such cells can be used for the above screening of the present invention.

Alternatively, the screening method of the present invention may comprise the following steps:
a) contacting a test compound with a polypeptide encoded by a polynucleotide selected from the group consisting of PRC 1-138;
b) detecting the biological activity of the polypeptide of step (a); and
c) selecting a compound that suppresses the biological activity of the polypeptide encoded by the polynucleotide selected from the group consisting of PRC 1-40 as compared to the biological activity detected in the absence of the test compound, or enhances the biological activity of the polypeptide encoded by the polynucleotide selected from the group consisting of PRC 41-138 as compared to the biological activity detected in the absence of the test compound.

A protein for use in the screening method of the present invention can be obtained as a recombinant protein using the nucleotide sequence of the marker gene. Based on the information regarding the marker gene and its encoded protein, one skilled in the art can select any biological activity of the protein as an index for screening and any suitable measurement method to assay for the selected biological activity.

Alternatively, the screening method of the present invention may comprise the following steps:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of PRC 1-138
b) measuring the expression or activity of said reporter gene; and
c) selecting the candidate compound that reduces the expression or activity level of said reporter gene when said marker gene is an up-regulated marker gene selected from the group consisting of PRC 1-40 as compared to a level in control, or that enhances the expression level of said reporter gene when said marker gene is a down-regulated marker gene selected from the group consisting of PRC 41-138, as compared to a level in control.

Suitable reporter genes and host cells are well known in the art. A reporter construct suitable for the screening method of the present invention can be prepared by using the transcriptional regulatory region of a marker gene. When the transcriptional regulatory region of the marker gene is known to those skilled in the art, a reporter construct can be prepared by using the previous sequence information. When the transcriptional regulatory region of the marker gene remains unidentified, a nucleotide segment containing the transcriptional regulatory region can be isolated from a genome library based on the nucleotide sequence information of the marker gene. In the present method, for example, a lebel detected in the absence of the test compound is preferable as the control expression lebel to be compared.

A compound isolated by the screening serves as a candidate for the development of drugs that inhibit or enhance the activity of the protein encoded by marker gene and can be applied to the treatment or prevention of PRC.

Moreover, compounds in which a part of the structure of the compound inhibiting or enhancing the activity of proteins encoded by marker genes is converted by addition, deletion and/or replacement are also included as the compounds obtainable by the screening method of the present invention.

When administrating a compound isolated by the method of the present invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredient contained in such a preparation makes a suitable dosage within the indicated range acquirable.

Examples of additives that can be admixed into tablets and capsules include, but are not limited to, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. When the unit-dose form is a capsule, a liquid carrier, such as an oil, can be further included in the above ingredients. Sterile composites for injection can be formulated following normal drug implementations using vehicles such as distilled water suitable for injection.

Physiological saline, glucose, and other isotonic liquids including adjuvants, such as D-sorbitol, D-mannnose, D-mannitol, and sodium chloride, can be used as aqueous solutions for injection. These can be used in conjunction with suitable solubilizers, such as alcohol, for example ethanol; polyalcohols, such as propylene glycol; and polyethylene glycol; and non-ionic surfactants, such as Polysorbate 80™ and HCO-50.

Sesame oil or soy-bean oil can be used as an oleaginous liquid, may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and/or an anti-oxidant. A prepared injection may be filled into a suitable ampoule.

Methods well known to those skilled in the art may be used to administer the pharmaceutical composition of the present invention to patients, for example as an intraarterial, intravenous, or percutaneous injection or as an intranasal, transbronchial, intramuscular or oral administration. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient; however one skilled in the art can suitably select them.

For example, although the dose of a compound that binds to a protein of the present invention and regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kgs of body-weight.

### Assessing the Prognosis of a Subject with PRC

The present invention also provides a method of assessing the prognosis of a subject with PRC including the step of comparing the expression of one or more PRC-associated genes in a test cell population to the expression of the same PRC-associated genes in a reference cell population derived from patients over a spectrum of disease stages. By comparing the gene expression of one or more PRC-associated genes in the test cell population and the reference cell population(s), or by comparing the pattern of gene expression over time in test cell populations derived from the subject, the prognosis of the subject can be assessed.

For example, a decrease in expression of one or more of PRC 41-138 compared to a expression in PIN or an increase of expression of one or more of PRC 1-40 compared to a expression in PIN indicates less favorable prognosis. An increase in expression of one or more of PRC 41-138 indicates a more favorable prognosis, and a decrease in expression of PRC 1-40 indicates a more favorable prognosis for the subject.

### Kits

The present invention also includes a PRC-detection reagent, e.g., a nucleic acid that specifically binds to or identifies one or more PRC nucleic acids, such as oligonucleotide sequences which are complementary to a portion of a PRC nucleic acid, or an antibody that bind to one or more proteins encoded by a PRC nucleic acid. The detection reagents may be packaged together in the form of a kit. The reagents are packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay may also be included in the kit. The assay format of the kit may be a Northern hybridization or a sandwich ELISA, both of which are known in the art.

For example, PRC detection reagent may be immobilized on a solid matrix such as a porous strip to form at least one PRC detection site. The measurement or detection region of the porous strip may include a plurality of sites, each containing a nucleic acid. A test strip may also contain sites for negative and/or positive controls. Alternatively, control sites may be located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of PRC present in the sample. The detection sites may be configured in any suitably detectable shape and are typically in the shape of a bar or dot spanning the width of a test strip.

Alternatively, the kit may contain a nucleic acid substrate array comprising one or more nucleic acids. The nucleic acids on the array specifically identify one or more nucleic acids sequences represented by PRC 1-138. The expression of 2, 3, 4, 5, 6, 7, 8, 9, 10, 15, 20, 25, 40 or 50 or more of the nucleic acids represented by PRC 1-138 are identified by virtue of the level of binding to an array test strip or chip. The substrate array can be on, e.g., a solid substrate, such as a “chip” described in U.S. Pat. No. 5,744,305, the contents of which are incorporated by reference herein in its entirety.

### Arrays and Pluralities

The present invention also includes a nucleic acid substrate array comprising one or more nucleic acids. The nucleic acids on the array specifically correspond to one or more nucleic acid sequences represented by PRC 1-138. The level of expression of 2, 3, 4, 5, 6, 7, 8, 9, 10, 15, 20, 25, 40 or 50 or more of the nucleic acids represented by PRC 1-138 may be identified by detecting nucleic acid binding to the array.

The present invention also includes an isolated plurality (i.e., a mixture of two or more nucleic acids) of nucleic acids. The nucleic acids may be in a liquid phase or a solid phase, e.g., immobilized on a solid support such as a nitrocellulose membrane. The plurality includes one or more of the nucleic acids represented by PRC 1-138. In various embodiments, the plurality includes 2, 3, 4, 5, 6, 7, 8, 9, 10, 15, 20, 25, 40 or 50 or more of the nucleic acids represented by PRC 1-138.

### Methods of Inhibiting PRC

The present invention further provides a method for treating or alleviating a symptom of PRC in a subject by decreasing the expression or activity of one or more of the PRC 1-40 (or the activity of its gene product) or increasing expression or activity of PRC 41-138 (or the activity of its gene product). Suitable therapeutic compounds can be administered prophylactically or therapeutically to a subject suffering from or at risk of (or susceptible to) developing PRC. Such subjects can be identified using standard clinical methods or by detecting an aberrant level of expression of one or more of the PRC 1-138 or aberrant activity of its gene product. In the context of the present invention, suitable therapeutic agents include, for example, inhibitors of cell cycle regulation, cell proliferation, and protein kinase activity.

The therapeutic method of the present invention includes the step of increasing the expression, function, or both of one or more gene products of genes whose expression is decreased (“down-regulated” or “under-expressed” genes”) in PRC cell relative to PIN cells of the same tissue type from which the PRC or PIN cells are derived. In these methods, the subject is treated with an effective amount of a compound that increases the amount of one or more of the under-expressed (down-regulated) genes in the subject. Administration can be systemic or local. Suitable therapeutic compounds include a polypeptide product of an under-expressed gene, a biologically active fragment thereof a nucleic acid encoding an under-expressed gene and having expression control elements permitting expression in the PRC cells; for example, an agent that increases the level of expression of such a gene endogenous to the PRC cells (i.e., which up-regulates the expression of the under-expressed gene or genes). Administration of such compounds counters the effects of aberrantly under-expressed gene or genes in the subject's prostate cells and improves the clinical condition of the subject.

Alternatively, the therapeutic method of the present invention may include the step of decreasing the expression, function, or both, of one or more gene products of genes whose expression is aberrantly increased (“up-regulated” or “over-expressed” gene”) in prostate cells. Expression may be inhibited in any of several ways known in the art. For example, expression can be inhibited by administering to the subject a nucleic acid that inhibits, or antagonizes, the expression of the over-expressed gene or genes, e.g., an antisense oligonucleotide or small interfering RNA which disrupts expression of the over-expressed gene or genes.

### Antisense Nucleic Acids

As noted above, antisense nucleic acids corresponding to the nucleotide sequence of PRC 1-40 can be used to reduce the expression level of the PRC 1-40. Antisense nucleic acids corresponding to PRC 1-40 that are up-regulated in PRC are useful for the treatment of PRC. Specifically, the antisense nucleic acids of the present invention may act by binding to the PRC 1-40 or mRNAs corresponding thereto, thereby inhibiting the transcription or translation of the genes, promoting the degradation of the mRNAs, and/or inhibiting the expression of proteins encoded by a nucleic acid selected from the group consisting of the PRC 1-40, finally inhibiting the function of the proteins. The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at least 80% or higher, more preferably at least 90% or higher, even more preferably at least 95% or higher over a span of at least 15 continuous nucleotides. Algorithms known in the art can be used to determine the homology.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by admixing it with a suitable base material which is inactive against the nucleic acid.

Also, as needed, the antisense nucleic acids of the present invention can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative of the present invention can be given to the patient by direct application onto the ailing site or by injection into a blood vessel so that it will reach the site of ailment. An antisense-mounting medium can also be used to increase durability and membrane-permeability. Examples include, but are, not limited to liposomes, poly-L-lysine, lipids, cholesterol, lipofectin or derivatives of these.

The dosage of the antisense nucleic acid derivative of the present invention can be adjusted suitably according to the patient's condition and used in desired amounts. For example, a dose range of 0.1 to 100 mg/kg, preferably 0.1 to 50 mg/kg can be administered.

The antisense nucleic acids of the present invention inhibit the expression of a protein of the present invention and are thereby useful for suppressing the biological activity of the protein of the invention. In addition, expression-inhibitors, comprising antisense nucleic acids of the present invention, are useful in that they can inhibit the biological activity of a protein of the present invention.

The antisense nucleic acids of present invention include modified oligonucleotides. For example, thioated oligonucleotides may be used to confer nuclease resistance to an oligonucleotide.

si RNA;

Also, a siRNA against a marker gene can be used to reduce the expression level of the marker gene. Herein term “siRNA” refers to a double stranded RNA molecule which prevents translation of a target mRNA. Standard techniques for introducing siRNA into the cell may be used, including those in which DNA is a template from which RNA is transcribed. In the context of the present invention, the siRNA comprises a sense nucleic acid sequence and an anti-sense nucleic acid sequence against an up-regulated marker gene, such as PRC 1-40. The siRNA is constructed such that a single transcript has both the sense and complementary antisense sequences from the target gene, e.g., a hairpin.

An siRNA of a PRC gene hybridizes to target mRNA and thereby decreases or inhibits production of the PRC polypeptides encoded by the gene by associating with the normally single-stranded mRNA transcript, thereby interfering with translation and thus, expression of the protein. Thus, siRNA molecules of the invention can be defined by their ability to hybridize specifically to mRNA of a gene selected from PRC 1-40 under stringent conditions. For the purposes of this invention the terms “hybridize” or “hybridize specifically” are used to refer the ability of two nucleic acid molecules to hybridize under “stringent hybridization conditions.” The phrase “stringent hybridization conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 50° C.

In the context of the present invention, an siRNA is preferably less than 500, 200, 100, 50, or 25 nucleotides in length. More preferably an siRNA is 19-25 nucleotides in length. In order to enhance the inhibition activity of the siRNA, nucleotide “u” can be added to 3′end of the antisense strand of the target sequence. The number of “u”s to be added is at least 2, generally 2 to 10, preferably 2 to 5. The added “u”s form single strand at the 3′end of the antisense strand of the siRNA.

An siRNA of a PRC gene can be directly introduced into the cells in a form that is capable of binding to the mRNA transcripts. In these embodiments, the siRNA molecules fo the invention are typically modified as described above for antisense molecules. Other modifications are also possible, for example, cholesterol-conjugated siRNAs have shown improved pharmacological properties (Song et al. Nature Med. 9:347-351 (2003):). Alternatively, a DNA encoding the siRNA may be carried in a vector.

Vectors may be produced, for example, by cloning a PRC gene target sequence into an expression vector having operatively-linked regulatory sequences flanking the sequence in a manner that allows for expression (by transcription of the DNA molecule) of both strands (Lee, N. S., Dohjima, T., Bauer, G., Li, H., Li, M. -J., Ehsani, A., Salvaterra, P., and Rossi, J. (2002) Expression of small interfering RNAs targeted against HIV-1 rev transcripts in human cells. Nature Biotechnology 20: 500-505.). An RNA molecule that is antisense to mRNA of a PRC-associated gene is transcribed by a first promoter (e.g., a promoter sequence 3′ of the cloned DNA) and an RNA molecule that is the sense strand for the mRNA of a PRC-associated gene is transcribed by a second promoter (e.g., a promoter sequence 5′ of the cloned DNA). The sense and antisense strands hybridize in vivo to generate siRNA constructs for silencing of the PRC-associated gene. Alternatively, the two constructs can be utilized to create the sense and anti-sense strands of a siRNA construct. Cloned PRC-associated genes can encode a construct having secondary structure, e.g., hairpins, wherein a single transcript has both the sense and complementary antisense sequences from the target gene.

A loop sequence consisting of an arbitrary nucleotide sequence can be located between the sense and antisense sequence in order to form the hairpin loop structure. Thus, the present invention also provides siRNA having the general formula 5′-[A]-[B]-[A′]-3′, wherein [A] is a ribonucleotide sequence corresponding to a sequence that specifically hybridizes to an mRNA or a cDNA of gene selected from PRC 1-40. In preferred embodiments, [A] is a ribonucleotide sequence corresponding a gene selected from PRC 1-40.

[B] is a ribonucleotide sequence consisting of 3 to 23 nucleotides, and

[A′] is a ribonucleotide sequence consisting of the complementary sequence of [A]. The region [A] hybridizes to [A′], and then a loop consisting of region [B] is formed. The loop sequence may be preferably 3 to 23 nucleotide in length. The loop sequence, for example, can be selected from group consisting of following sequences (http://www.ambion.com/techlib/tb/tb_{—}506.html). Furthermore, loop sequence consisting of 23 nucleotides also provides active siRNA (Jacque, J. -M., Triques, K., and Stevenson, M. (2002) Modulation of HIV-1 replication by RNA interference. Nature 418 : 435-438.).

CCC, CCACC or CCACACC: Jacque, J. M, Triques, K., and Stevenson, M (2002) Modulation of HIV-1 replication by RNA interference. Nature, Vol. 418: 435-438.

UUCG: Lee, N. S., Dohjima, T., Bauer, G., Li, H., Li, M.-J., Ehsani, A., Salvaterra, P., and Rossi, J. (2002) Expression of small interfering RNAs targeted against HIV-1 rev transcripts in human cells. Nature Biotechnology 20: 500-505. Fruscoloni, P., Zamboni, M., and Tocchini-Valentini, G. P. (2003) Exonucleolytic degradation of double-stranded RNA by an activity in Xenopus laevis germinal vesicles. Proc. Natl. Acad. Sci. USA 100(4): 1639-1644.

UUCAAGAGA: Dykxhoorn, D. M., Novina, C. D., and Sharp, P. A. (2002) Killing the messenger: Short RNAs that silence gene expression. Nature Reviews Molecular Cell Biology 4: 457-467.

Accordingly, the loop sequence can be selected from group consisting of, CCC, UUCG, CCACC, CCACACC, and UUCAAGAGA. Preferable loop sequence is UUCAAGAGA (“ttcaagaga” in DNA).

The nucleotide sequence of suitable siRNAs can be designed using an siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. don't recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene to evaluate.

The regulatory sequences flanking the PRC gene sequences can be identical or different, such that their expression can be modulated independently, or in a temporal or spatial manner. siRNAs are transcribed intracellularly by cloning the PRC gene templates, respectively, into a vector containing, e.g., a RNA pol III transcription unit from the small nuclear RNA (snRNA) U6 or the human H1 RNA promoter. For introducing the vector into the cell, transfection-enhancing agent can be used. FuGENE (Rochediagnostices), Lipofectamin 2000 (Invitrogen), Oligofectamin (Invitrogen), and Nucleofactor (Wako pure Chemical) are useful as the transfection-enhancing agent.

The antisense oligonucleotide or siRNA of the present invention inhibits the expression of a polypeptide of the present invention and is thereby useful for suppressing the biological activity of a polypeptide of the invention. Also, expression-inhibitors, comprising the antisense oligonucleotide or siRNA of the invention, are useful in the point that they can inhibit the biological activity of the polypeptide of the invention. Therefore, a composition comprising an antisense oligonucleotide or siRNA of the present invention is useful for treating or preventing a PRC.

### Antibodies:

Alternatively, function of one or more gene products of the genes over-expressed in PRC can be inhibited by administering a compound that binds to or otherwise inhibits the function of the gene products. For example, the compound is an antibody which binds to the over-expressed gene product or gene products.

The present invention refers to the use of antibodies, particularly antibodies against a protein encoded by an up-regulated marker gene, or a fragment of such an antibody. As used herein, the term “antibody” refers to an immunoglobulin molecule having a specific structure, that interacts (i.e., binds) only with the antigen that was used for synthesizing the antibody (i.e., the gene product of an up-regulated marker) or with an antigen closely related thereto. Furthermore, an antibody may be a fragment of an antibody or a modified antibody, so long as it binds to one or more of the proteins encoded by the marker genes. For instance, the antibody fragment may be Fab, F(ab′)₂, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. Such modification methods are conventional in the field.

Alternatively, an antibody may comprise as a chimeric antibody having a variable region derived from a nonhuman antibody and a constant region derived from a human antibody, or a humanized antibody, comprising a complementarity determining region (CDR) derived from a nonhuman antibody, the frame work region (FR) derived from a human antibody and the constant region. Such antibodies can be prepared by using known technologies. Humanization can be performed by substituting rodent CDRs or CDR sequences for the corresponding sequences of a human antibody (see e.g., Verhoeyen et al., Science 239:1534-1536 (1988)). Accordingly, such humanized antibodies are chimeric antibodies, wherein substantially less than an intact human variable domain has been substituted by the corresponding sequence from a non-human species.

Fully human antibodies comprising human variable regions in addition to human framework and constant regions can also be used. Such antibodies can be produced using various techniques known in the art. For example in vitro methods involve use of recombinant libraries of human antibody fragments displayed on bacteriophage (e.g., Hoogenboom & Winter, J. Mol. Biol. 227:381 (1991), Similarly, human antibodies can be made by introducing of human immunoglobulin loci into transgenic animals, e.g., mice in which the endogenous immunoglobulin genes have been partially or completely inactivated. This approach is described, e.g., in U.S. Pat. Nos. 6,150,584, 5,545,807; 5,545,806; 5,569,825; 5,625,126; 5,633,425; 5,661,016.

Cancer therapies directed at specific molecular alterations that occur in cancer cells have been validated through clinical development and regulatory approval of anti-cancer drugs such as trastuzumab (Herceptin) for the treatment of advanced breast cancer, imatinib methylate (Gleevec) for chronic myeloid leukemia, gefitinib (Iressa) for non-small cell lung cancer (NSCLC), and rituximab (anti-CD20 mAb) for B-cell lymphoma and mantle cell lymphoma (Ciardiello F, Tortora G. A novel approach in the treatment of cancer: targeting the epidermal growth factor receptor. Clin Cancer Res. 2001 Oct;7(10):2958-70. Review.; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J Med. 2001 Mar. 15;344(11):78392; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15;101(2):420-424; Fang G, Kim C N, Perkins C L, Ramadevi N, Winton E, Wittmann S and Bhalla K N. (2000). Blood, 96, 2246-2253). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56; Klejman A, Rushen L, Morrione A, Slupianek A and Skorski T. (2002). Oncogene, 21, 5868-5876). Therefore, future cancer treatments will probably involve combining conventional drugs with target-specific agents aimed at different characteristics of tumor cells such as angiogenesis and invasiveness.

These modulatory methods can be performed ex vivo or iii vitro (e.g., by culturing the cell with the agent) or, alternatively, in vivo (e.g., by administering the agent to a subject). The methods involve administering a protein or combination of proteins or a nucleic acid molecule or combination of nucleic acid, molecules as therapy to counteract aberrant expression of the differentially expressed genes or aberrant activity of their gene products.

Diseases and disorders that are characterized by increased (relative to a subject not suffering from the disease or disorder) expression levels or biological activity in PIN of genes and gene products, respectively, may be treated with therapeutics that antagonize (i.e., reduce or inhibit) activity of the over-expressed gene or genes. Therapeutics that antagonize activity can be administered therapeutically or prophylactically.

Accordingly, therapeutics that may be utilized in the context of the present invention including, e.g., (i) a polypeptide of the over-expressed or under-expressed gene or genes, or analogs, derivatives, fragments or homologs thereof; (ii) antibodies to the overexpressed gene or gene products; (iii) nucleic acids encoding the under-expressed gene or gene s; (iv) antisense nucleic acids or nucleic acids that are “dysfunctional” (i.e., due to a heterologous insertion within the nucleic acids of one or more over-expressed gene or genes); (v) small interfering RNA (siRNA); or (vi) modulators (i.e., inhibitors, agonists and antagonists that alter the interaction between an over/under-expressed polypeptide and its binding partner). The dysfunctional antisense molecules are utilized to “knockout” endogenous function of a polypeptide by homologous recombination (see, e.g., Capecchi, Science 244: 1288-1292 1989).

Diseases and disorders that are characterized by decreased expression levels or biological activity in PIN of gene and gene products may be treated with therapeutics that increase (i.e., are agonists to) activity. Therapeutics that up-regulate activity may be administered in a therapeutic or prophylactic manner. Therapeutics that may be utilized include, but are not limited to, a polypeptide (or analogs, derivatives, fragments or homologs thereof) or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Prophylactic administration occurs prior to the manifestation of overt clinical symptoms of disease, such that a disease or disorder is prevented or, alternatively, delayed in its progression.

Therapeutic methods of the present invention may include the step of contacting a cell with an agent that modulates one or more of the activities of the gene products of the differentially expressed genes. Examples of agent that modulates protein activity include, but are not limited to, a nucleic acids, proteins, a naturally-occurring cognate ligands of such proteins, peptides, a peptidomimetics, and other small molecule. For example, a suitable agent may stimulate one or more protein activities of one or more differentially under-expressed genes.

### Vaccinating Against Prostate Cancer:

The present invention also relates to a method of treating or preventing PRC in a subject comprising the step of administering to said subject a vaccine comprising a polypeptide encoded by a nucleic acid selected from the group consisting of PRC 1-40 or an immunologically active fragment of said polypeptide, or a polynucleotide encoding such a polypeptide or fragment thereof. Administration of the polypeptide induces an anti-tumor immunity in a subject. To induce anti-tumor immunity, a polypeptide encoded by a nucleic acid selected from the group consisting of PRC 1-40 or an immunologically active fragment of said polypeptide, or a polynucleotide encoding such a polypeptide or fragment thereof is administered to subject in need thereof. The polypeptide or the immunologically active fragments thereof are useful as vaccines against PRC. In some cases, the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, a vaccine against PRC refers to a substance that has the ability to induce anti-tumor immunity upon inoculation into animals. According to the present invention, polypeptides encoded by a nucleic acid selected from the group consisting of PRC 1-40 or fragments thereof were suggested to be HLA-A24 or HLA-A*0201 restricted epitopes peptides that may induce potent and specific immune response against PRC cells expressing PRC 1-40. Thus, the present invention also encompasses method of inducing anti-tumor immunity using the polypeptides. In general, antitumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is determined to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. Specifically a foreign substance that enters the living body is presented to T cells and B cells by the action of antigen presenting cells (APCs). T cells that respond to the antigen presented by the APCs in an antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to a T cell via an APC, and detecting the induction of CTLs. Furthermore, APCs have the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTLs using dendritic cells (DCs) as the APC is well known in the art. DCs are a representative APCs having the strongest CTL-inducing action among APCs. In this method, the test polypeptide is initially contacted with DCs, and then the DCs are contacted with T cells. Detection of T cells having cytotoxic effects against the cells of interest after the contact with DC shows that the test polypeptide has an activity of inducing the cytotoxic T cells. Activity of CTLs against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DCs, peripheral blood mononuclear cells (PBMCs) may also be used as the APC. The induction of CTLs has been reported to be enhanced by culturing PBMCs in the presence of GM-CSF and IL-4. Similarly, CTLs have been shown to be induced by culturing PBMCs in the presence of keyhole limpet hemocyanin (KLH) and IL-7.

Test polypeptides confirmed to possess CTL-inducing activity by these methods are deemed to be polypeptides having DC activation effect and subsequent CTL-inducing activity. Therefore, polypeptides that induce CTLs against tumor cells are useful as vaccines against tumors. Furthermore, APCs that have acquired the ability to induce CTLs against tumors through contact with the polypeptides are also useful as vaccines against tumors. Furthermore, CTLs, that have acquired cytotoxicity due to presentation of the polypeptide antigens by APCs can also be used as vaccines against tumors. Such therapeutic methods for tumors using anti-tumor immunity due to APCs and CTLs are referred to as cellular immunotherapy.

Generally, when using a polypeptide for cellular immunotherapy, efficiency of the CTL-induction is known to be increased by combining a plurality of polypeptides having different structures and contacting them with DCs. Therefore, when stimulating DCs with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide is deemed to have the ability to induce anti-tumor immunity.

Anti-tumor immunity is induced by administering the vaccine of this invention, and the induction of anti-tumor immunity enables treatment and prevention of PRC. Therapy against cancer or prevention of the onset of cancer includes any of the following steps, such as inhibition of the growth of cancerous cells, involution of cancer, and suppression of occurrence of cancer. A decreases in mortality and mortality of individuals having cancer, decrease in the levels of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's test, the Mann-Whitney U-test, or ANOVA may be used for statistical analysis.

The above-mentioned protein having immunological activity or a vector encoding the protein may be combined with an adjuvant. An adjuvant refers to a compound that enhances the immune response against the protein when administered together (or successively) with the protein having immunological activity. Exemplary adjuvants include, but are not limited to, cholera toxin, salmonella toxin, alum, and such, but are not limited thereto. Furthermore, the vaccine of this invention may be combined appropriately with a pharmaceutically acceptable carrier. Examples of such carriers includes sterilized water, physiological saline, phosphate buffer, culture fluid, and such. Furthermore, the vaccine may contain as necessary, stabilizers, suspensions, preservatives, surfactants, and such. The vaccine can be administered systemically or locally. Vaccine administration can be performed by single administration, or boosted by multiple administrations.

When using an APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APCs or CTLs, the cells may be administered to the subject. APCs can be also induced by introducing a vector encoding the polypeptide into PBMCs ex vivo. APCs or CTLs induced in vitro can be cloned prior to administration. By cloning and growing cells having high activity of damaging target cells, cellular immunotherapy can be performed more effectively. Furthermore, APCs and CTLs isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

### Pharmaceutical Compositions for Inhibiting PRC

In the context of the present invention, suitable pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. Preferably, administration is intravenous. The formulations are optionally packaged in discrete dosage units.

Pharmaceutical formulations suitable for oral administration include capsules, cachets or tablets, each containing a predetermined amount of active ingredient. Suitable formulations also include powders, granules, solutions, suspensions and emulsions. The active ingredient is optionally administered as a bolus electuary or paste. Tablets and capsules for oral administration may contain conventional excipients, such as binding agents, fillers, lubricants, disintegrant and/or wetting agents. A tablet may be made by compression or molding, optionally with one or more formulational ingredients. Compressed tablets may be prepared by compressing in a suitable machine the active ingredients in a free-flowing form, such as a powder or granules, optionally mixed with a binder, lubricant, inert diluent, lubricating, surface active and/or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), and/or preservatives. The tablets may optionally be formulated so as to provide slow or controlled release of the active ingredient therein. A package of tablets may contain one tablet to be taken on each of the month.

Formulations suitable for parenteral administration include aqueous and non-aqueous sterile injection solutions, optionally contain anti-oxidants, buffers, bacteriostats and solutes which render the formulation isotonic with the blood of the intended recipient; as well as aqueous and non-aqueous sterile suspensions including suspending agents and/or thickening agents. The formulations may be presented in unit dose or multi-dose containers, for example as sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations suitable for rectal administration include suppositories with standard carriers such as cocoa butter or polyethylene glycol. Formulations suitable for topical administration in the mouth, for example buccally or sublingually, include lozenges, containing the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray, a dispersible powder or in the form of drops. Drops may be formulated with an aqueous or non-aqueous base also comprising one or more dispersing agents, solubilizing agents and/or suspending agents.

For administration by inhalation the compounds can be conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, for example, as capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants and/or preservatives.

It should be understood that in addition to the ingredients particularly mentioned above, the formulations of this invention may include other agents conventional in the art with regard to the type of formulation in question. For example, formulations suitable for oral administration may include flavoring agents.

Preferred unit dosage formulations contain an effective dose, as recited below, or an appropriate fraction thereof, of the active ingredient.

For each of the aforementioned conditions, the compositions, e.g., polypeptides and organic compounds, can be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity. In any event, appropriate and optimum dosages may be routinely calculated by those skilled in the art, taking into consideration the above-mentioned factors.

Aspects of the present invention are described in the following examples, which are not intended to limit the scope of the invention described in the claims. The following examples illustrate the identification and characterization of genes differentially expressed in PRC or PIN cells.

### BEST MODE FOR CARRYING OUT THE INVENTION

The invention will be further described in the following examples, which do not limit the scope of the invention described in the claims.

### Example 1

### General Methods

### Patients and Tissue Samples

Tissue samples were obtained with informed consent from 26 cancer patients undergoing radical prostatectomy. All surgical specimens were at clinical stages T2a-T3a with or without N1, and their Gleason scores were 5-9. Histopathological diagnoses were made by a single pathologist before LMM. All samples were embedded in TissueTek OCT medium (Sakura, Tokyo, Japan) immediately after surgical resection and stored at −80° C. until use. From among the 26 resected tissues, 20 cancers and 10 high-grade PINs had sufficient amounts and quality of RNA for microarray analysis.

### Laser Microbeam Microdissection and T7-Based RNA Amplification

LMM and T7based RNA amplification were performed as described previously. Prostate tumor cells, prostatic intraepithelial neoplasia cells and normal prostatic ductal epithelial cells were isolated selectively using the EZ cut system with a pulsed ultraviolet narrow beam-focus laser (SL Microtest GmbH, Germany) in accordance with the manufacturer's protocols. After DNase treatment, total RNAs were subjected to two rounds of T7based amplification, which yielded 50-100 μg of aRNA from each sample. Then 2.5 μg aliquots of aRNA from PRC or PIN cells and from normal prostatic ductal epithelial cells were labeled by reverse transcription with Cy5-dCTP (tumor cells) or Cy3-dCTP (normal cells) (Amersham Biosciences, Buckinghamshire, UK), as described previously (Ono et al. 2000).

cDNA Microarray Analysis and Acquisition of Data

We fabricated a genome-wide cDNA microarray with 23,040 cDNAs selected from the UniGene database (build #131) of the National Center for Biotechnology Information (NCBI). Construction, hybridization, washing, and scanning were carried out according to methods described previously (Ono et al. 2000). Signal intensities of Cy3 and Cy5 from the 23,040 spots were quantified and analyzed by substituting backgrounds, using ArrayVision software (Imaging Research, Inc., St. Catharines, Ontario, Canada). Subsequently, the fluorescent intensities of Cy5 (tumor) and Cy3 (control) for each target spot were adjusted so that the mean Cy3/Cy5 ratio of 52 housekeeping genes was equal to one. Because data derived from low signal intensities are less reliable, we determined a cut-off value on each slide (Ono et al. 2000) and excluded genes from further analysis when both Cy3 and Cy5 dyes yielded signal intensities lower than the cut-off. For other genes, we calculated the Cy5/Cy3 ratio using the raw data of each sample.

### Identification of Genes That Were Up- or Down-Regulated from PINs to PRC

We identified genes with changed expression in 20 PRC and 10 PINs according to the following criteria: 1) genes for which we were able to obtain expression data in more than 50% of the cases examined; and 2) genes whose expression ratio was more than 3.0 in prostate cancers and between 0.5 and 2.0 in PINs (defined as up-regulated genes) or genes whose expression ratio was less than 0.33 in cancers and between 0.5 and 2.0 in PINs (defined as down-regulated genes) in more than 50% of informative cases.

### Immunohistochemistry

Formalin-fixed and paraffin-embedded prostatic tumor sections were immunostained using a mouse anti-APOD (apolipoprotein D) monoclonal antibody (NEOMARKERS, Fremont, Calif.) for APOD expression. Prostate cancer tissues included PRC cells, PIN cells and normal protstatic epithelium heterogeneously. Deparaffinized tissue sections were placed in 10 mM citrate buffer, pH 6.0, and heated to 108° C. in an autoclave for 15 minutes for antigen retrieval. Sections were incubated with a 1:10 dilution or a 1:100 dilution of primary antibody for APOD, respectively, in a humidity chamber for an hour at room temperature, and developed with peroxidase labeled-dextran polymer followed by diaminobenzidine (DAKO Envision Plus System; DAKO Corporation, Carpinteria, Calif.). Sections were counterstained with hematoxylin. For negative controls, primary antibody was omitted.

### Example 2

### Identification of Genes Up- or Down-Regulated During Malignant Transformation from PINs to Prostate Cancers

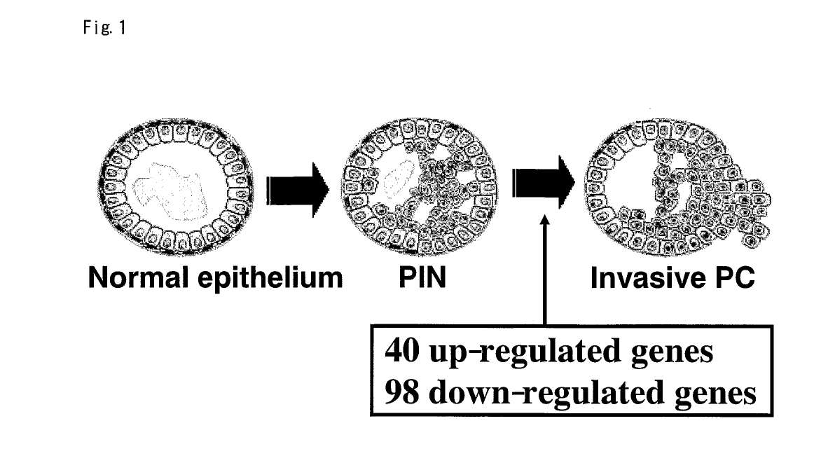

We focused on differential expression patterns between PINs and PRC to search for genes likely to be involved in the transition from non-invasive precursor PINs to malignant cancers (FIG. 1). Comparing the expression profiles of 20 PRC with those of 10 PINs, we identified 40 up-regulated genes (Table 1) and 98 down-regulated genes (Table 2). The list included POV1, CDKN2C, APOD, FASN, and VWF as up-regulated, and ITGB2, LAMB2, PLAU and TIMP1 as down-regulated; the altered genes might be involved with cell adhesion or motility in invasive PRC cells. Some of the later are associated with cell adhesion and proteinase activity, suggesting that their expression changes may contribute to the invasive phenotype by abolishing ductal structures during the transition from PIN to PRC.

TABLE 1
Up-regulated genes in the transition from PIN to PRC

| | | | |
|---|---|---|---|
| Accession No. | Hs. | Symbol | Title |

function known

| | | | | |
|---|---|---|---|---|
| 1 | X12433 | 99364 | ABHD2 | abhydrolase domain containing 2 |
| 2 | AF039018 | 135281 | ALP | alpha-actinin-2-associated LIM protein |
| 3 | H61951 | 12152 | APMCF1 | APMCF1 protein |
| 4 | J02611 | 75736 | APOD | apolipoprotein D |
| 5 | AA633487 | 108708 | CAMKK2 | calcium/calmodulin-dependent protein |
| | | | | kinase kinase 2, beta |
| 6 | AI357641 | 4854 | CDKN2C | cyclin-dependent kinase inhibitor 2C (p18, |
| | | | | inhibits CDK4) |
| 7 | NM_004938 | 153924 | DAPK1 | death-associated protein kinase 1 |
| 8 | NM_004405 | 419 | DLX2 | distal-less homeo box 2 |
| 9 | T78186 | 241565 | DNMT3A | DNA (cytosine-5-)-methyltransferase 3 |
| | | | | alpha |
| 10 | W94051 | 336678 | DTNA | dystrobrevin, alpha |
| 11 | M16967 | 30054 | F5 | coagulation factor V (proaccelerin, labile |
| | | | | factor) |
| 12 | U29344 | 83190 | FASN | fatty acid synthase |
| 13 | AF100143 | 6540 | FGF13 | fibroblast growth factor 13 |
| 14 | D14446 | 107 | FGL1 | fibrinogen-like 1 |
| 15 | BE747327 | 7644 | HIST1H1C | histone 1, H1c |
| 16 | BG163483 | 76907 | HSPC002 | HSPC002 protein |
| 17 | AF064493 | 4980 | LDB2 | LIM domain binding 2 |
| 18 | U21128 | 79914 | LUM | lumican |
| 19 | U07620 | 151051 | MAPK10 | mitogen-activated protein kinase 10 |
| 20 | NM_002465 | 169849 | MYBPC1 | myosin binding protein C, slow type |
| 21 | AI767296 | 123655 | NPR3 | natriuretic peptide receptor C/guanylate |
| | | | | cyclase C |
| 22 | X76770 | 49007 | PAPOLA | poly(A) polymerase alpha |
| 23 | AF045584 | 18910 | POV1 | prostate cancer overexpressed gene 1 |
| 24 | AI298501 | 21192 | SDK1 | sidekick homolog 1 (chicken) |
| 25 | AB020335 | 181300 | SEL1L | sel-1 suppressor of lin-12-like (C. elegans) |
| 26 | BE735822 | 75069 | SHMT2 | serine hydroxymethyltransferase 2 |
| | | | | (mitochondrial) |
| 27 | N21096 | 99291 | STXBP6 | syntaxin binding protein 6 (amisyn) |
| 28 | AF091395 | 367689 | TRIO | triple functional domain (PTPRF |
| | | | | interacting) |
| 29 | AK000235 | 31608 | TRPM4 | transient receptor potential cation channel, |
| | | | | subfamily M, member 4 |
| 30 | NM_000552 | 110802 | VWF | von Willebrand factor |

function unknown

| | | | | |
|---|---|---|---|---|
| 31 | N66442 | 135971 | | ESTs |
| 32 | BE274422 | 380933 | | Homo sapiens mRNA; cDNA |
| | | | | DKFZp586O1224 |
| 33 | AI304487 | 171443 | | Homo sapiens, clone IMAGE: 3354344, |
| | | | | mRNA, partial cds |
| 34 | AA830405 | 403857 | | Homo sapiens, clone IMAGE: 5932767, |
| | | | | mRNA |
| 35 | D14657 | 81892 | KIAA0101 | KIAA0101 gene product |
| 36 | W63676 | 356547 | LOC129642 | hypothetical protein BC016005 |
| 37 | AW295100 | 5957 | LOC201562 | hypothetical protein LOC201562 |
| 38 | AL137707 | 103422 | LOC220115 | hypothetical protein LOC220115 |
| 39 | AI057614 | 293845 | LOC89944 | hypothetical protein BC008326 |
| 40 | AW972144 | 422113 | MGC30006 | hypothetical protein MGC30006 |

TABLE 2
Down-regulated genes in the transition from PIN to PRC

| | | | |
|---|---|---|---|
| Accession No. | Hs. | Symbol | Title |

function known

| | | | | |
|---|---|---|---|---|
| 41 | AI827230 | 374481 | APCDD1 | adenomatosis polyposis coli down- |
| | | | | regulated 1 |
| 42 | BF965257 | 74120 | APM2 | adipose specific 2 |
| 43 | AA156854 | 114309 | APOL1 | apolipoprotein L, 1 |
| 44 | NM_004024 | 460 | ATF3 | activating transcription factor 3 |
| 45 | M94345 | 82422 | CAPG | capping protein (actin filament), gelsolin- |
| | | | | like |
| 46 | AF035752 | 139851 | CAV2 | caveolin 2 |
| 47 | D13639 | 75586 | CCND2 | cyclin D2 |
| 48 | M16445 | 89476 | CD2 | CD2 antigen (p50), sheep red blood cell |
| | | | | receptor |
| 49 | AI750036 | 22116 | CDC14B | CDC14 cell division cycle 14 homolog B |
| | | | | (S. cerevisiae) |
| 50 | AK021865 | 173380 | CKIP-1 | CK2 interacting protein 1; HQ0024c |
| | | | | protein |
| 51 | X15880 | 108885 | COL6A1 | collagen, type VI, alpha 1 |
| 52 | L16510 | 297939 | CTSB | cathepsin B |
| 53 | U03688 | 154654 | CYP1B1 | cytochrome P450, family 1, subfamily B, |
| | | | | polypeptide 1 |
| 54 | M62401 | 82568 | CYP27A1 | cytochrome P450, family 27, subfamily A, |
| | | | | polypeptide 1 |
| 55 | X90579 | 166079 | CYP3A5P2 | cytochrome P450, family 3, subfamily A, |
| | | | | polypeptide 5 pseudogene 2 |
| 56 | X93920 | 180383 | DUSP6 | dual specificity phosphatase 6 |
| 57 | NM_001421 | 151139 | ELF4 | E74-like factor 4 (ets domain transcription |
| | | | | factor) |
| 58 | AF275945 | 116651 | EVA1 | epithelial V-like antigen 1 |
| 59 | AW300770 | 61265 | FAM3D | family with sequence similarity 3, member D |
| 60 | D84239 | 111732 | FCGBP | Fc fragment of IgG binding protein |
| 61 | AF182316 | 234680 | FER1L3 | fer-1-like 3, myoferlin (C. elegans) |
| 62 | NM_001924 | 80409 | GADD45A | growth arrest and DNA-damage-inducible, |
| | | | | alpha |
| 63 | W91908 | 6079 | GALNAC4 | B cell RAG associated protein |
| | | | S-6ST | |
| 64 | AA666119 | 92287 | GBP3 | guanylate binding protein 3 |
| 65 | NM_000165 | 74471 | GJA1 | gap junction protein, alpha 1, 43 kDa |
| | | | | (connexin 43) |
| 66 | J03004 | 77269 | GNAI2 | guanine nucleotide binding protein (G |
| | | | | protein), alpha inhibiting activity |
| | | | | polypeptide 2 |
| 67 | J03817 | 301961 | GSTM1 | glutathione S-transferase M1 |
| 68 | M33906 | 198253 | HLA-DQA1 | major histocompatibility complex, class II, |
| | | | | DQ alpha 1 |
| 69 | NM_018950 | 110309 | HLA-F | major histocompatibility complex, class I, F |
| 70 | U26726 | 1376 | HSD11B2 | hydroxysteroid (11-beta) dehydrogenase 2 |
| 71 | BF793633 | 180919 | ID2 | inhibitor of DNA binding 2, dominant |
| | | | | negative helix-loop-helix protein |
| 72 | AV646610 | 34853 | ID4 | inhibitor of DNA binding 4, dominant |
| | | | | negative helix-loop-helix protein |
| 73 | M15395 | 83968 | ITGB2 | integrin, beta 2 |
| 74 | U25138 | 93841 | KCNMB1 | potassium large conductance calcium- |
| | | | | activated channel, subfamily M, beta |
| | | | | member 1 |
| 75 | AB012955 | 129867 | KIP2 | DNA-dependent protein kinase catalytic |
| | | | | subunit-interacting protein 2 |
| 76 | X72760 | 90291 | LAMB2 | laminin, beta 2 (laminin S) |
| 77 | Y00711 | 234489 | LDHB | lactate dehydrogenase B |
| 78 | M36682 | 621 | LGALS3 | lectin, galactoside-binding, soluble, 3 |
| | | | | (galectin 3) |
| 79 | L13210 | 79339 | LGALS3BP | lectin, galactoside-binding, soluble, 3 |
| | | | | binding protein |
| 80 | X03444 | 377973 | LMNA | lamin A/C |
| 81 | AA779709 | 7457 | MAGE-E1 | MAGE-E1 protein |
| 82 | L08895 | 78995 | MEF2C | MADS box transcription enhancer factor 2, |
| | | | | polypeptide C |
| 83 | AF017418 | 104105 | MEIS2 | Meis1, myeloid ecotropic viral integration |
| | | | | site 1 homolog 2 (mouse) |
| 84 | AF203032 | 198760 | NEFH | neurofilament, heavy polypeptide 200 kDa |
| 85 | M12267 | 75485 | OAT | ornithine aminotransferase (gyrate atrophy) |
| 86 | AW051593 | 189999 | P2RY5 | purinergic receptor P2Y, G-protein |
| | | | | coupled, 5 |
| 87 | BG028573 | 64056 | PAK1 | p21/Cdc42/Rac1-activated kinase 1 |
| | | | | (STE20 homolog, yeast) |
| 88 | BF969355 | 8364 | PDK4 | pyruvate dehydrogenase kinase, isoenzyme 4 |
| 89 | AA253194 | 303125 | PIGPC1 | p53-induced protein PIGPC1 |
| 90 | M22430 | 76422 | PLA2G2A | phospholipase A2, group IIA (platelets, |
| | | | | synovial fluid) |
| 91 | D00244 | 77274 | PLAU | plasminogen activator, urokinase |
| 92 | X56134 | 297753 | RPLP2 | ribosomal protein, large P2 |
| 93 | W73992 | 132792 | SDCCAG43 | serologically defined colon cancer antigen |
| | | | | 43 |
| 94 | AW965789 | 66450 | SENP1 | sentrin/SUMO-specific protease |
| 95 | AF029082 | 184510 | SFN | stratifin |
| 96 | U44403 | 75367 | SLA | Src-like-adaptor |
| 97 | AV705470 | 380991 | SNF1LK | SNF1-like kinase |
| 98 | Y08110 | 101657 | SORL1 | sortilin-related receptor, L(DLR class) A |
| | | | | repeats-containing |
| 99 | BE439695 | 160483 | STOM | stomatin |
| 100 | AB042646 | 94785 | TGIF2 | TGFB-induced factor 2 (TALE family |
| | | | | homeobox) |
| 101 | NM_003241 | 2387 | TGM4 | transglutaminase 4 (prostate) |
| 102 | U21847 | 82173 | TIEG | TGFB inducible early growth response |
| 103 | M12670 | 5831 | TIMP1 | tissue inhibitor of metalloproteinase 1 |
| 104 | AA837002 | 9741 | TJP4 | tight junction protein 4 (peripheral) |
| 105 | M35252 | 84072 | TM4SF3 | transmembrane 4 superfamily member 3 |
| 106 | M19309 | 73980 | TNNT1 | troponin T1, skeletal, slow |
| 107 | W72411 | 137569 | TP73L | tumor protein p73-like |
| 108 | H99016 | 171501 | USP11 | ubiquitin specific protease 11 |
| 109 | AF077197 | 74669 | VAMP5 | vesicle-associated membrane protein 5 |
| | | | | (myobrevin) |
| 110 | AW137980 | 115659 | VIK | vav-1 interacting Kruppel-like protein |
| 111 | D88154 | 103665 | VILL | villin-like |
| 112 | M92843 | 343586 | ZFP36 | zinc finger protein 36, C3H type, homolog |
| | | | | (mouse) |
| 113 | BF055342 | 326801 | ZNF6 | zinc finger protein 6 (CMPX1) |

function unknown

| | | | | |
|---|---|---|---|---|
| 114 | AI769569 | 112472 | | ESTs |
| 115 | AW510657 | 156044 | | ESTs |
| 116 | BF111819 | 21470 | | ESTs |
| 117 | T79422 | 119237 | | ESTs |
| 118 | AI304862 | 12867 | | ESTs |
| 119 | AA705222 | 119880 | | ESTs |
| 120 | AA768607 | 122926 | | ESTs |
| 121 | AI052358 | 131741 | | ESTs |
| 122 | AW888225 | 250723 | | ESTs, Weakly similar to hypothetical |
| | | | | protein FLJ20378 [Homo sapiens] |
| 123 | BF223679 | 118747 | | Homo sapiens cDNA FLJ33407 fis, clone |
| | | | | BRACE2010535. |
| 124 | AI821113 | 292781 | | Homo sapiens cDNA FLJ36327 fis, clone |
| | | | | THYMU2005748. |
| 125 | AL360198 | 22870 | | Homo sapiens mRNA full length insert |
| | | | | cDNA clone EUROIMAGE 34988. |
| 126 | AL050204 | 28540 | | Homo sapiens mRNA; cDNA |
| | | | | DKFZp586F1223 (from clone |
| | | | | DKFZp586F1223) |
| 127 | AV733210 | 367688 | | Homo sapiens, clone IMAGE: 4794726, |
| | | | | mRNA |
| 128 | U57961 | 181304 | 13CDNA73 | hypothetical protein CG003 |
| 129 | AL050289 | 7446 | C6orf4 | chromosome 6 open reading frame 4 |
| 130 | AW956111 | 79404 | D4S234E | DNA segment on chromosome 4 (unique) |
| | | | | 234 expressed sequence |
| 131 | AK001021 | 22505 | FLJ10159 | hypothetical protein FLJ10159 |
| 132 | R43725 | 98927 | FLJ13993 | hypothetical protein FLJ13993 |
| 133 | D42047 | 82432 | KIAA0089 | KIAA0089 protein |
| 134 | NM_014766 | 75137 | KIAA0193 | KIAA0193 gene product |
| 135 | AA921341 | 3610 | KIAA0205 | KIAA0205 gene product |
| 136 | AB007903 | 113082 | KIAA0443 | KIAA0443 gene product |
| 137 | BF431643 | 15420 | KIAA1500 | KIAA1500 protein |
| 138 | AA706316 | 32343 | ZD52F10 | hypothetical gene ZD52F10 |

### Example 3

### Immunohistochemistry

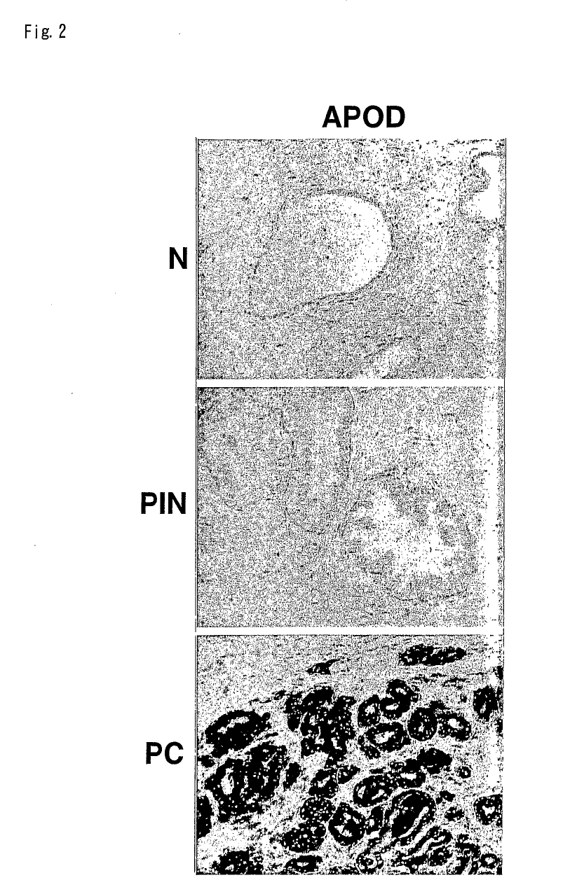

To validate the gene expression pattern in the transition from PIN to PRC, we performed immunohistochemical analysis of the genes differentially expressed in the transition from PIN to PRC in our data. In general, prostate cancer tissues includes PRC cells, PIN cells and normal prostatic epithelium heterogenously, and we compared the staining pattern of each kinds of cells associated with prostatic carcinogenesis on the same tissues from the same patient. As shown in FIG. 2, apolipoprotein D (APOD) was abundantly expressed in PRC cells while PINs and normal prostatic epithelium from the same patient had no or very weak expression of APOD protein. The results implicate this expression profile analysis is highly reliable.

### INDUSTRIAL APPLICABILITY

The gene-expression analysis of PRC and PIN described herein, obtained through a combination of laser-capture dissection and genome-wide cDNA microarray, has identified specific genes as targets for cancer prevention and therapy. Based on the expression of a subset of these differentially expressed genes, the present invention provides a molecular diagnostic markers for diagnosing a predisposition to developing PRC.

The methods described herein are also useful in the identification of additional molecular targets for prevention, and treatment of PRC. The data reported herein add to a comprehensive understanding of PRC, facilitate development of novel diagnostic strategies, and provide clues for identification of molecular targets for therapeutic drugs and preventative agents. Such information contributes to a more profound understanding of prostatic tumorigenesis, and provide indicators for developing novel strategies for diagnosis, treatment, and ultimately prevention of PRC.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

Abate-Shen, C., and Shen, M. M. Molecular genetics of prostate cancer. Genes & Dev., 14: 2410-2434, 2000.
Bostwick, D. G. Prostatic intraepithelial neoplasia. Curr. Urol. Rep., 1: 65-70, 2000.
DeMarzo, A. M., Nelson, W. G., Isaacs, W. B., and Epstein, J. I. Pathological and molecular aspects of prostate cancer. Lancet, 361: 955-964, 2003.
Gronberg, H. Prostate cancer epidemiology. Lancet, 361: 859-64, 2003.
McNeal, J. E., and Bostwick, D. G. Intraductal dysplasia: a premalignant lesion of the prostate. Hum. Pathol., 17: 64-71, 1986.
Montironi R, Mazzucchelli R, Scarpelli M. Precancerous lesions and conditions of the prostate: from morphological and biological characterization to chemoprevention. Ann. N. Y. Acad. Sci., 963: 169-184, 2002.
Nelson, G. N., and Wilding, G. Prostate cancer prevention agent: criteria and pipeline for candidate chemoprevention agents. Urology, 57, 56-63, 2001.
Ono, K., Tanaka, T., Tsunoda, T., Kitahara, O., Kihara, C., Okamoto, A., Ochiai, K., Takagi, T., and Nakamura, Y. Alterations of gene expression during colorectal carcinogenesis revealed by cDNA microarrays after laser-capture microdissection of tumor tissues and normal epithelia. Cancer Res., 60: 5007-5011, 2000.
Steiner, M. S. High grade prostatic intraepithelial neoplasia is a disease. Curr. Urol. Rep., 2: 195-198, 2001.
van der Kwast, T. H., Lopes, C., Santonja, C., Pihl, C. G., Neetens, I., Martikainen, P., Di Lollo, S., Bubendorf, L., Hoedemaeker, R. F.; Members of the pathology committee of the European Randomised Study of Screening for Prostate Cancer. Guidelines for processing and reporting of prostatic needle biopsies. J Clin. Pathol., 56:336-40, 2003.
   Emmert-Buck, M. R., Bonner, R. F., Smith, P. D., Chuaqui, R. F., Zhuang, Z., Goldstein, S. R., Weiss, R. A., and Liotta, L. A. Laser capture microdissection. Science, 274: 998-1001, 1996.
   Han M, Partin A W, Piantadosi S, Epstein J I, Walsh P C. J Urol, 166: 416-419, 2001.

1. A method of diagnosing a predisposition to developing PRC in a subject, comprising determining a level of expression of a PRC-associated gene in a patient derived biological sample, wherein an increase or decrease of said level compared to expression level of said gene in PIN indicates that said subject is at risk of developing PRC.

2. The method of claim 1, wherein said PRC-associated gene is selected from the group consisting of PRC 1-40, wherein an increase in said level compared to a level in PIN indicates said subject is at risk of developing PRC.

3. The method of claim 2, wherein said increase is at least 10% greater than said level in PIN.

4. The method of claim 1, wherein said PRC-associated gene is selected from the group consisting of PRC 41-138, wherein a decrease in said level compared to a level in PIN indicates said subject is at risk of developing PRC.

5. The method of claim 4, wherein said decrease is at least 10% lower than said level in PIN.

6. The method of claim 1, wherein said method further comprises determining said level of expression of a plurality of PRC associated genes.

7. The method of claim 1, wherein the expression level is determined by any one method select from group consisting of:
(a) detecting the mRNA of the PRC-associated genes,
(b) detecting the protein encoded by the PRC-associated genes, and
(c) detecting the biological activity of the protein encoded by the PRC-associated genes.

8. The method of claim 1, wherein said level of expression is determined by detecting hybridization of a PRC-associated gene probe to a gene transcript of said patient-derived biological sample.

9. The method of claim 8, wherein said hybridization step is carried out on a DNA array.

10. The method of claim 1, wherein said biological sample comprises an epithelial cell.

11. The method of claim 1, wherein said biological sample comprises prostate cancer cell.

12. The method of claim 8, wherein said biological sample comprises an epithelial cell from a PRC.

13. A PRC reference expression profile, comprising a pattern of gene expression of two or more genes selected from the group consisting of PRC 1-138.

14. A PRC reference expression profile, comprising a pattern of gene expression of two or more genes selected from the group consisting of PRC 1-40.

15. A PRC reference expression profile, comprising a pattern of gene expression of two or more genes selected from the group consisting of PRC 41-138.

16. A method of screening for a compound for treating or preventing PRC, said method comprising the steps of:
a) contacting a test compound with a polypeptide encoded by PRC 1-138;
b) detecting the binding activity between the polypeptide and the test compound; and
c) selecting a compound that binds to the polypeptide.

17. A method of screening for a compound for treating or preventing PRC, said method comprising the steps of:
a) contacting a candidate compound with a cell expressing one or more marker genes, wherein the one or more marker genes is selected from the group consisting of PRC 1-138; and
b) selecting a compound that reduces the expression level of one or more marker genes selected from the group consisting of PRC 1-40, or elevates the expression level of one or more marker genes selected from the group consisting of PRC 41-138.

18. The method of claim 17, wherein said test cell comprises a prostate cancer cell.

19. A method of screening for a compound for treating or preventing PRC, said method comprising the steps of:
a) contacting a test compound with a polypeptide encoded by selected from the group consisting of PRC 1-138;
b) detecting the biological activity of the polypeptide of step (a); and
c) selecting a compound that suppresses the biological activity of the polypeptide encoded by PRC 1-40 in comparison with the biological activity detected in the absence of the test compound, or enhances the biological activity of the polypeptide encoded by PRC 41-138 in comparison with the biological activity detected in the absence of the test compound.

20. A method of screening for compound for treating or preventing PRC, said method comprising the steps of:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of PRC 1-138
b) measuring the expression or activity of said reporter gene; and
c) selecting a compound that reduces the expression or activity level of said reporter gene when said marker gene is an up-regulated marker gene selected from the group consisting of PRC 1-40, as compared to a level in control or that enhances the expression level of said reporter gene when said marker gene is a down-regulated marker gene selected from the group consisting of PRC 41-138, as compared to a level in control.

21. A kit comprising a detection reagent which binds to two or more nucleic acid sequences selected from the group consisting of PRC 1-138.

22. An array comprising a nucleic acid which binds to two or more nucleic acid sequences selected from the group consisting of PRC 1-138.

23. A method of treating or preventing PRC in a subject comprising administering to said subject an antisense composition, said composition comprising a nucleotide sequence complementary to a coding sequence selected from the group consisting of PRC 1-40.

24. A method of treating or preventing PRC in a subject comprising administering to said subject a siRNA composition, wherein said composition reduces the expression of a nucleic acid sequence selected from the group consisting of PRC 1-40.

25. A method of treating or preventing PRC in a subject comprising the step of administering to said subject a pharmaceutically effective amount of an antibody or fragment thereof that binds to a protein encoded by any one gene selected from the group consisting of PRC 1-40.

26. A method of treating or preventing PRC in a subject comprising administering to said subject a vaccine comprising a polypeptide encoded by a nucleic acid selected from the group consisting of PRC 1-40 or an immunologically active fragment of said polypeptide, or a polynucleotide encoding the polypeptide.

27. A method of treating or preventing PRC in a subject comprising administering to said subject a compound that increases the expression or activity of PRC 41-138.

28. A method of treating or preventing PRC in a subject, said method comprising the step of administering a compound that is obtained by the method according to any one of claims 16-20.

29. A method of treating or preventing PRC in a subject comprising administering to said subject a pharmaceutically effective amount of polynucleotide select from group consisting of PRC 41-138, or polypeptide encoded by thereof.

30. A composition for treating or preventing PRC, said composition comprising a pharmaceutically effective amount of an antisense polynucleotide or small interfering RNA against a polynucleotide select from group consisting of PRC 1-40 as an active ingredient, and a pharmaceutically acceptable carrier.

31. A composition for treating or preventing PRC, said composition comprising a pharmaceutically effective amount of an antibody or fragment thereof that binds to a protein encoded by any one gene selected from the group consisting of PRC 1-40 as an active ingredient, and a pharmaceutically acceptable carrier.

32. A composition for treating or preventing PRC, said composition comprising a pharmaceutically effective amount of the compound selected by the method of any one of claims 16-20 as an active ingredient, and a pharmaceutically acceptable carrier.
